(19) United States
(12) Patent Application Publication
(10) Pub. No.: US 2014/0194962 A1
(43) Pub. Date: Jul. 10, 2014

(54) Aesculapius Probe System

(71) Applicant: Scott Douglas Ulrich, Apple Valley, MN (US)

(72) Inventor: Scott Douglas Ulrich, Apple Valley, MN (US)

(21) Appl. No.: 13/694,798

(22) Filed: Jan. 7, 2013

(51) Int. Cl. A61N 1/05

(52) U.S. Cl. CPC A61N 1/0504
USPC 607/116

(57) ABSTRACT

An Aesculapius Probe System (device) which is a portable method of controlling surgically installed probes (electrodes) requiring a controlled voltage source. The Aesculapius Probe System incorporates electrical control of Ag (silver) electrodes to inject silver (Ag+) ions to the target infection or point of injury. Portability is accomplished using surface mount technologies (electrical components) and DC coin-cell or thin-cell battery technologies.

### TECHNICAL FIELD

Various embodiments described herein relate to an apparatus, system and method for healing. More particularly, this invention relates to an apparatus and method for topical healing and relates to an apparatus and method for healing sub dermal or subcutaneous infections and diseases. The present invention relates generally to medical devices, and more particularly but not by limitation to surgically implanted voltage controlled probes (electrodes) used in infection control and accelerated healing of the human (mammals) body.

### BACKGROUND

Numerous procedures and therapies have been attempted or utilized in connection with treatment of wounds, including application to the wound of various stimulation and/or medicaments to aid the natural body healing functions. It has been found, however, that at least some known stimulations or medicaments cannot be utilized for a particular treatment or in connection with particular individuals, and it has also been found that some wounds, including chronic wounds, resist healing even with aggressive and intense treatment. Conventional medical probes (electrodes) requiring a voltage source and surgical installation often immobilized a patient for the duration of the treatment due to the necessity of a constant voltage source and conductor connections; this would often require hospitalization.

In the past, it has been suggested that the healing process might be promoted or accelerated through use of electrical stimulation, and several methods for effecting such treatment have been proposed, with some such methods having been heretofore utilized with varying degrees of success. Among the more successful has been bone growth stimulation for promoting bone healing.

The relationship between direct current electricity and cellular mitosis and cellular growth has become better understood during the latter half of the twentieth century. Weiss, in Weiss, Daryl S., et. al., Electrical Stimulation and Wound Healing, Arch Dermatology, 126:222 (February 1990), points out that living tissues naturally possess direct current electropotentials that regulate, at least in part, the wound healing process. Following tissue damage, a current of injury is generated that is thought to trigger biological repair. This current of injury has been extensively documented in scientific studies. It is believed that this current of injury is instrumental in ensuring that the necessary cells are drawn to the wound location at the appropriate times during the various stages of wound healing. Localized exposure to low levels of electrical current that mimic this naturally occurring current of injury has been shown to enhance the healing of soft tissue wounds in both human subjects and animals. It is thought that these externally applied fields enhance, augment, or take the place of the naturally occurring biological field in the wound environment, thus fostering the wound healing process.

Weiss continues to explain, in a summary of the scientific literature, that intractable ulcers have demonstrated accelerated healing and skin wounds have resurfaced faster and with better tensile properties following exposure to electrical currents. Dayton and Palladino, in Dayton, Paul D., and Palladino, Steven J., Electrical Stimulation of Cutaneous Ulcerations—A Literature Review, Journal of the American Podiatric Medical Association, 79(7):318 (July 1989), also state that the alteration of cellular activity with externally applied currents can positively or negatively influence the status of a healing tissue, thereby directing the healing process to a desired outcome.

Furthermore, research conducted by Rafael Andino during his graduate tenure at the University of Alabama at Birmingham, also demonstrated that the presence of electrical fields (in this case induced by the application of pulsating electromagnetic fields) dramatically accelerated the healing rates of wounds created in an animal model. This research found that the onset and duration of the first two phases of the wound healing process, the inflammatory and proliferative phases, had been markedly accelerated in the treated wounds while the volume of collagen which had been synthesized by the fibroblasts was also markedly increased in the treated wounds. This resulted in the wounds healing in a much shorter amount of time. Similar findings from other researchers can be found in other wound healing literature.

Even though electrical stimulation has been suggested to promote healing of soft tissue wounds, to date, no known method has been suggested that has proved to be completely successful, perhaps due to the many and varied parameters of the many problems presented by such injuries.

Thus, as can be appreciated from the foregoing, various procedures, or methods, have been heretofore suggested that utilize many differing parameters. It is felt, however, that procedures, or methods, are still needed that can be demonstrated to enhance healing of soft tissue, and diseases found below the skin. In addition, probe selection (material) and current levels used for selected application used in healing were often non-optimal and resulted in unintended collateral cell damage.

### SUMMARY OF THE INVENTION

### BRIEF DESCRIPTION OF THE DRAWINGS

FIG. 1 is block diagram view of an apparatus for applying healing current to site on injury, according to an example embodiment.

FIG. 2 is a graphical plot of delivered-current base on voltage and resistance selection, according to an example embodiment.

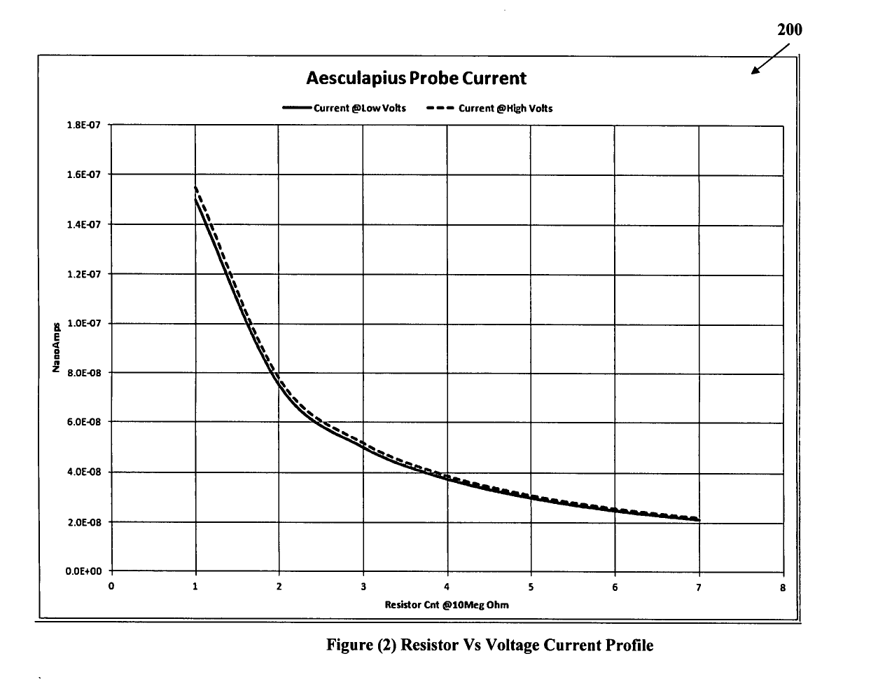

FIG. 3 is an electrical schematic of the apparatus shown in FIG. 1, according to an example embodiment.

FIG. 4(a) is prior art perspective view (example) of a non-portable stimulus system, according to an example embodiment.

FIG. 4(b) is a perspective view of an installed Aesculapius Probe System placed in a treatment position, according to an example embodiment.

FIG. 5 is a layout of a flex circuit (circuit board) of the apparatus shown in FIG. 6 shows a diagrammatic representation of a computer system, within which a set of instructions for causing the machine to perform any one or more of the methodologies discussed herein can be executed.

FIG. 7 is a Method Flow drawing of an instruction set, according to an example embodiment.

### DETAILED DESCRIPTION

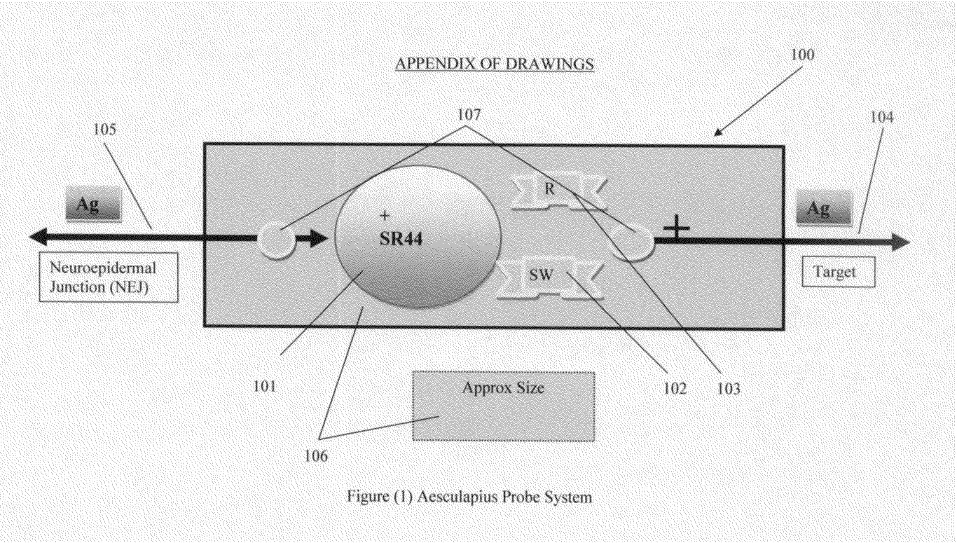

FIG. 1 is block diagram view of an apparatus 100 for applying current to an area, according to an example embodiment. The apparatus 100 includes a printed circuit board 106. On the printed circuit board is a power source 101, such as a coin-cell battery. One embodiment, the button battery 101 is a silver oxide chemistry battery that has about a 50% greater than the thin alkaline chemistry usually produces a flat discharge characteristic. Namely a constant voltage output. The voltage of silver oxide chemistry battery is relatively constant. In addition the silver oxide chemistry batteries have about a 50% greater capacity than alkaline chemistry batteries. In addition to the portability, the silver oxide technology is closer to the bio-voltage levels desired. In one embodiment, the button battery 101 used is an Energizer silver oxide button battery commonly known as an SR 44. The battery produces approximately 1.35 V of relatively or substantially constant output voltage. In other embodiments various battery technologies are useable as long as proper current and voltage levels are achieved.

The apparatus 100 also includes a resistor 103 which is used to bring down the amperage of the current produced by the button battery or power source 101. In one embodiment the resistor is selected to bring down the amperage of the circuit to approximately a range of 100-200 nanoamperes to approximate bio-current levels and minimize collateral cell damage. In another embodiment, different valued resistance can be used. The apparatus 100 also includes a first probe or wire 105 and a second probe wire 104. The first probe 105 is a negative probe or wire. The second probe 104 is a positive probe or wire. In one embodiment both the first probe or wire 105 and the second probe or wire 104 are made of silver (Ag 0.999). The apparatus 100 also includes a switch 102 which may be used to switch the polarity between the probes to mimic bio-messages of either healing or injury. 105, 104.

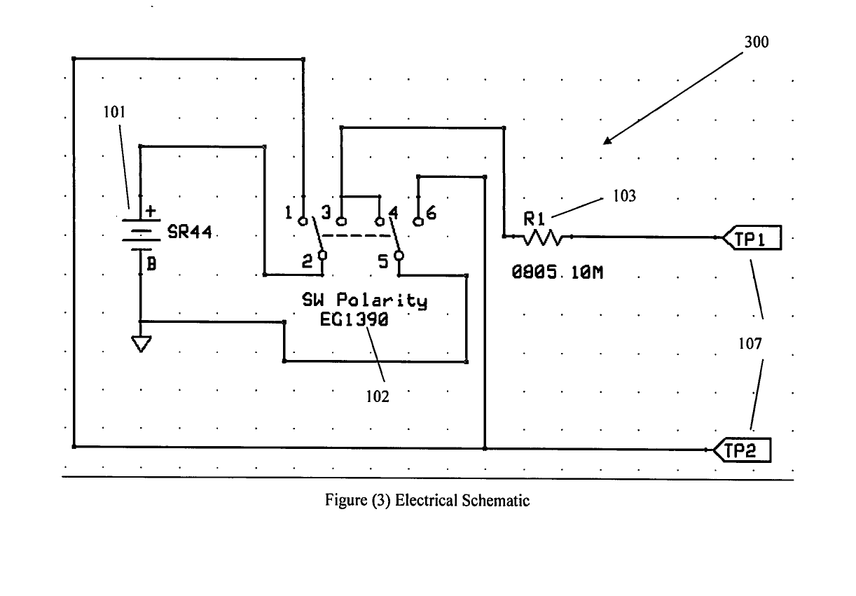

FIG. 3 is an electrical schematic of the apparatus 100 shown in FIG. 1, according to an example embodiment. As shown in FIG. 3), the apparatus 100 includes a power source 101. In one embodiment the power source is an SR 44 silver oxide chemistry button battery. Of course it should be noted that other power sources can be used. The apparatus also includes an electrical resistor 103, which is labeled R1 in FIG. (3). The apparatus also includes a polarity switch 102. The polarity switch 102 includes a first set of contacts (see contact 1 in contact 4) and a second set of contacts (see contact three in contact six). The polarity switch 102 is a dual action switch. When the contacts 1 and 4 are connected to contacts 2 and 5, the polarity at probes or wires 105, 104 are in a first state. When the contacts 3 and 6 are attached to contacts 2 and 5, the polarity of the probes 104, 105 are switch to a second state. Thus, the polarity at the probes 104, 105 can be switched as needed. In another embodiment the switch can be replaced by a microprocessor to control current profiles that more precisely match current of injury and current of healing for select application of injury and individual variability.

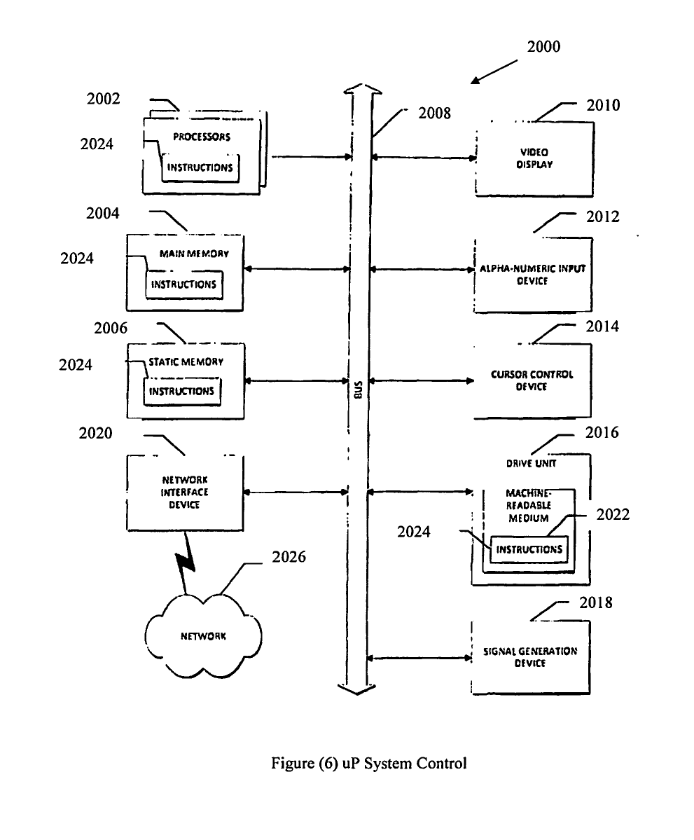

FIG. 5 is an enlarged top view of the probe layout on a flex circuit or printed circuit board 106, according to an example embodiment. The device 100 includes a position for the power source 101. The printed circuit board 106 also includes a position for the resistor 103 as well as the contact points for the polarity switch 102. The printed circuit board also includes output pads 107 correlated to the output 105 and the output 104. Probes or electrodes can be attached to the output pads 107 to form the first probe 105 and the second probe 104. The positive electrode of silver (Ag) in this embodiment is located at the point of injury. The positive silver (Ag) electrode will produce silver ions (Ag+) at the site where it is installed. The Silver (Ag+) ions under the small current of the probe System are “injected” further than just diffusion from a static Ag (silver) wire or electrode alone.

The Positive (+) end of probe can be used for killing infection. Sphere of influence will be approximately ½″ dia. from Ag wire (round wire). Note: Ag at the positive pole will kill or deactivate every type of bacterial without collateral damage. Silver (Ag) is effective even against anti-biotic strains of bacterial and fungus infections. Silver ion (Ag+) effect will produce accelerated healing time at the point of injury. Finally, Ag+ will suspend cancerous mitoses.

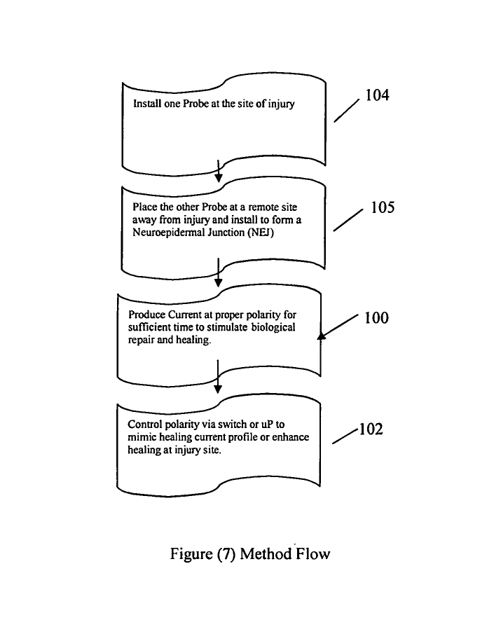

FIG. 7 is a flow chart of a method for applying a current to a subcutaneous area, according to an example embodiment. The method includes placing one probe at a site to be healed. The probe 104, is typically placed at the site to be healed. In some instances this may require an operation to place the probe 104 near the injured site. Once the probe is placed, the other probe is placed at a second site remote from the area to be healed to create a Neuropidermal Junction (NEJ). The polarity is selected so that it will stimulate biological repair. The controller system 100 can then be attached to the silver electrode implant. The printed circuit board 106 can then be attached to the probes 105, 104 and the power source 101 can be enabled to begin the process or method. The populated circuit board 106 is sufficiently small so that it can be easily attached to a body, such as an animal body or the human body allowing mobility. Once attached, the patient is free to move about within some limits. In another embodiment of the invention, the printed circuit board can be provided with a timer or a microprocessor or controller to monitor the probes as well as the condition of a patient and to automatically switch the polarity of the probes to create current profiles.

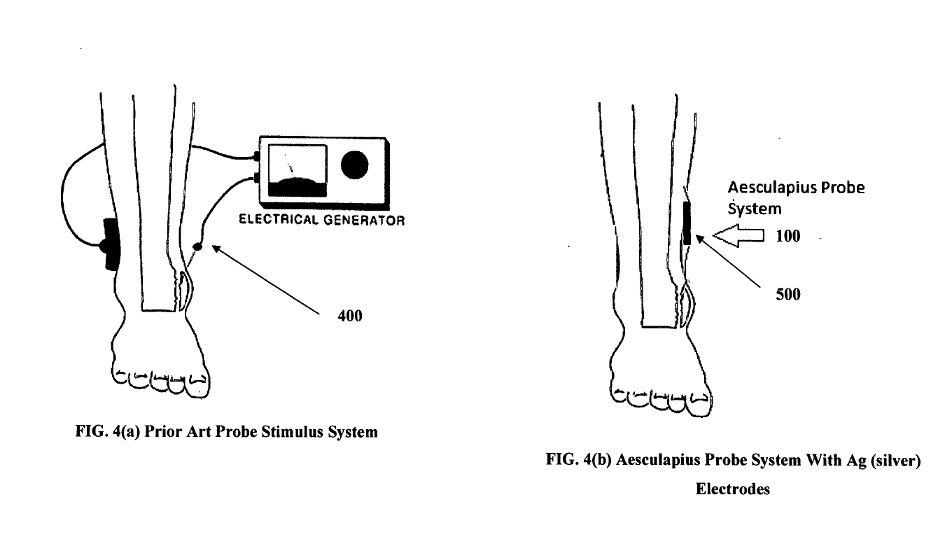

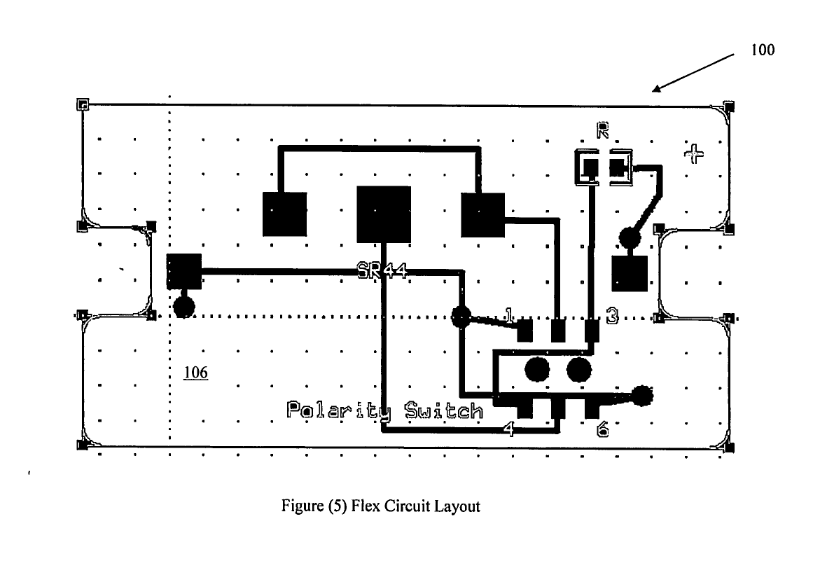

FIG. 4(b) is a perspective view of a flex circuit (printed circuit board) placed in a treatment position, according to an example embodiment. As can be seen, the printed circuit board 106 is bandaged or otherwise strapped and position on an appendage of the patient. The patient can freely move about within reason, while the apparatus 100 is in a treatment position with the wires and probes 105, 104 in place.

FIG. 6 shows a diagrammatic representation of a computer system 2000, within which a set of instructions for causing the machine to perform any one or more of the methodologies discussed herein can be executed. In various example embodiments, the machine operates as a standalone device or can be connected (e.g., networked) to other machines. In a networked deployment, the machine can operate in the capacity of a server or a client machine in a server-client network environment, or as a peer machine in a peer-to-peer (or distributed) network environment. The machine can be a personal computer (PC), a tablet PC, a set-top box (STB), a Personal Digital Assistant (PDA), a cellular telephone, a portable music player (e.g., a portable hard drive audio device such as a Moving Picture Experts Group Audio Layer 3 (MP3) player, a web appliance, a network router, a switch, a bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 2000 includes a processor or multiple processors 2002 (e.g., a central processing unit (CPU), a graphics processing unit (GPU), arithmetic logic unit or all), and a main memory 2004 and a static memory 2006, which communicate with each other via a bus 2008. The computer system 2000 can further include a video display unit 2010 (e.g., a liquid crystal displays (LCD) or a cathode ray tube (CRT)). The computer system 2000 also includes an alphanumeric input device 2012 (e.g., a keyboard), a cursor control device 2014 (e.g., a mouse), a disk drive unit 2016, a signal generation device 2018 (e.g., a speaker) and a network interface device 2020.

The disk drive unit 2016 includes a computer-readable medium 2022 on which is stored one or more sets of instructions and data structures (e.g., instructions 2024) embodying or utilized by any one or more of the methodologies or functions described herein. The instructions 2024 can also reside, completely or at least partially, within the main memory 2004 and/or within the processors 2002 during execution thereof by the computer system 2000. The main memory 2004 and the processors 2002 also constitute machine-readable media.

The instructions 2024 can further be transmitted or received over a network 2026 via the network interface device 2020 utilizing any one of a number of well-known transfer protocols (e.g., Hyper Text Transfer Protocol (HTTP), CAN, Serial, or Modbus).

While the computer-readable medium 2022 is shown in an example embodiment to be a single medium, the term “computer-readable medium” should be taken to include a single medium or multiple media (e.g., a centralized or distributed database, and/or associated caches and servers) that store the one or more sets of instructions and provide the instructions in a computer readable form. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, tangible forms and signals that can be read or sensed by a computer. Such media can also include, without limitation, hard disks, floppy disks, flash memory cards, digital video disks, random access memory (RAMs), read only memory (ROMs), and the like.

When a computerized method, discussed above, is programmed into a memory of a general purpose computer, the computer and instructions form a special purpose machine. The instructions, when programmed into a memory of a general purpose computer, are in the form of a non transitory set of instructions.

The example embodiments described herein can be implemented in an operating environment comprising computer-executable instructions (e.g., software) installed on a computer, in hardware, or in a combination of software and hardware. Modules as used herein can be hardware or hardware including circuitry to execute instructions. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software programs for implementing the present method(s) can be written in any number of suitable programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Perl, UNIX Shell, Visual Basic or Visual Basic Script, Virtual Reality Markup Language (VRML), ColdFusion™ or other compilers, assemblers, interpreters or other computer languages or platforms.

A machine readable medium that includes an instruction set, according to an example embodiment. The machine-readable medium that provides instructions that, when executed by a machine, cause the machine to perform operations associated with controlling the various components of the healing apparatus 100. When a healing apparatus 100 is provided with a microcontroller or other processor, it capable of forming a system. The machine-readable medium can also be used to instruct the processor to vary current levels in the healing apparatus 100 to enhance healing. It should also be noted that in other systems a plurality of healing apparatus 100 can be implemented at substantially the same time to several healing sites within a patient. In other words, a single processor can be used to communicate and control several of the healing apparatus.

The present disclosure refers to instructions that are received at a memory system. Instructions can include an operational command, e.g., read, write, erase, refresh, etc., an address at which an operational command should be performed, and the data, if any, associated with a command. The instructions can also include error correction data.

This has been a detailed description of some exemplary embodiments of the invention(s) contained within the disclosed subject matter. Such invention(s) may be referred to, individually and/or collectively, herein by the term “invention” merely for convenience and without intending to limit the scope of this application to any single invention or inventive concept if more than one is in fact disclosed. The detailed description refers to the accompanying drawings that form a part hereof and which shows by way of illustration, but not of limitation, some specific embodiments of the invention, including a preferred embodiment. These embodiments are described in sufficient detail to enable those of ordinary skill in the art to understand and implement the inventive subject matter. Other embodiments may be utilized and changes may be made without departing from the scope of the inventive subject matter. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for healing comprising:
placing a first probe in a healing location;
placing a second probe in another location; and
applying power to the first probe to produce a current in a first probe by way of a portable device.

2. The method for healing of claim 1 wherein the first probe and the second probe are surgically installed.

3. The method healing of claim 1 wherein the power is applied by a controlled voltage source.

4. The method for healing of claim 1 wherein a processor controls a current profile delivered via the first probe and the second probe.

5. The method for healing of claim 4 wherein the current profile replicates a current of injury and a current of healing.

6. The method for healing of claim 1 further comprising switching the polarity of the first probe and the second probe.

7. The method for healing of claim 6 wherein a processor controls a switching mechanism to control a current profile delivered by the first probe and the second probe.

8. The method for healing of claim 1 further comprising limiting the current to deliver current at biological levels to a target location proximate at least one of the first probe and the second probe.

9. A portable apparatus comprising:
a circuit board;
a power source coupled to the circuit board;
a first probe attached to the power source;
a second probe attached to the power source; and
a switching mechanism operatively coupled to the power source, the first probe and the second probe to switch the polarity of the first probe and the second probe, the first probe and the second probe delivering current to a target location at a level to replicate biological levels that promote healing.

10. The portable apparatus of claim 9 wherein the power source is a silver oxide battery.

11. The portable apparatus of claim 9 wherein the first probe and the second probe are made of a material that produces silver (Ag+) ions.

12. The portable apparatus of claim 9 wherein at least one of the first probe and the second probe are made of a material that produces silver (Ag+) ions.

13. The portable apparatus of claim 9 wherein the printed circuit board is a flexible circuit.

14. The portable apparatus of claim 9 wherein the first probe and the second probe are made of a material that produces silver (Ag+) ions.

15. The portable apparatus of claim 9 wherein the first probe and the second probe deliver current to a target location at a level to replicate biological levels associated with an injury.

16. The portable apparatus of claim 9 further comprising a processor, the processor controlling the switching mechanism to produce a current profile that replicates biological levels associated with injury and associated with healing.

17. The portable apparatus of claim 9 wherein biological levels of current are in the range of 100 to 200 nanoamperes.

18. The portable apparatus of claim 9 wherein the first probe and the second probe include portions adapted to be surgically implanted.

19. A portable method of silver ion (Ag+) injection to target infection or point of injury.

20. The portable method of claim 19 further comprising:
providing a power source;
providing a first probe attached to the power source; and
providing a second probe attached to the power source, the first and second probe producing silver ions (Ag+) at current levels associated with biological healing levels.
